(19) United States
(12) Patent Application Publication
(10) Pub. No.: US 2023/0313279 A1
(43) Pub. Date: Oct. 5, 2023

(54) METHODS OF DETERMINING THE LOCATION OF AN ANALYTE IN A BIOLOGICAL SAMPLE USING A PLURALITY OF WELLS

(71) Applicant: 10x Genomics, Inc., Pleasanton, CA (US)

(72) Inventors: Stefania Giacomello, Stockholm (SE); Paulius Mielinis, Stockholm (SE); Marlon Stoeckius, Stockholm (SE)

(73) Assignee: 10x Genomics, Inc., Pleasanton, CA (US)

(21) Appl. No.: 18/023,963

(22) PCT Filed: Sep. 16, 2021
(86) PCT No.: PCT/US2021/050625

§ 371 Date: Feb. 28, 2023

Related U.S. Application Data
(60) Provisional application No. 63/079,153, filed on Sep. 16, 2020.

(51) Int. Cl. C12Q 1/6841; C12Q 1/6874

(52) U.S. Cl. CPC C12Q 1/6841; C12Q 1/6874; C12Q 2600/16; C12Q 1/6876

(57) ABSTRACT

Provided herein are methods of determining a location of an analyte in a biological sample and devices that include a plurality of wells, where a well of the plurality of wells comprises a surface comprising a plurality of capture probes.

### CROSS-REFERENCE TO RELATED APPLICATION

This application claims priority under 35 U.S.C. § 119(e) to U.S. Patent Application Ser. No. 63/079,153, filed on Sep. 16, 2020, the entire contents of which are hereby incorporated by reference.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

The use of a single spatially-barcoded array can allow the determination of the position and contents of single cells from a parent biological sample. After permeabilization of the sample, the biological contents of the cell flow freely into the surrounding solution and can passively or actively migrate to the spatially-barcoded array beneath. However, the spatial resolution of the array can be limited by the thermodynamic diffusion of sample contents after permeabilization. Active migration methods (e.g., electrophoresis) can limit, but not eliminate, this diffusion.

### SUMMARY

Provided herein are methods and means for localizing cells from a biological sample into a spatially-barcoded well. A cell can be permeabilized or lysed within the spatially-barcoded well, thereby ensuring that the entirety of the cell's contents remain within the spatially-barcoded well for binding to capture probes (and subsequent analysis). In some examples, a biological sample can be sectioned into an approximately cell-sized constituent sections by pressing the sample into an array of spatially-barcoded wells.

The containment of cells and their contents within a single well leads to higher efficiency in the capture as analytes do not have to diffuse through a relatively large volume of solution before binding to a capture domain of a capture probe. Additionally, capturing the transcriptome of a cell leads to higher sensitivity and spatial resolution in assays. The use of wells with individual populations of capture probes opens the possibility of running multi-omics assays on a single slide. Affixing capture probes to the walls of a well can additionally support 3D spatial barcoding of the capture probes, allowing a third dimension of spatial resolution. Additionally, the use of a well mimics a microdroplet environment. Spatially restricting the volume of a reaction (using the methods and substrates described herein) can result in an increase in the speed and efficiency of reaction kinetics, while decreasing the use of assay reagents.

Provided herein are methods of determining a location of an analyte in a biological sample that include: (a) disposing portions of a biological sample into a plurality of wells, where a well of the plurality of wells comprises a surface comprising a plurality of capture probes, where a capture probe of the plurality comprises a spatial barcode and a capture domain; (b) releasing the analyte from the biological sample, where the analyte is specifically bound by the capture domain of the capture probe; and (c) determining (i) a sequence corresponding to the analyte or a complement thereof, and (ii) a sequence corresponding to the spatial barcode or a complement thereof, and using the sequences of (i) and (ii) to determine the location of the analyte in the biological sample. In some embodiments of any of the methods described herein, the substrate comprises about 1 million wells to about 5 million wells.

In some embodiments of any of the methods described herein, each of the plurality of wells is a flat-bottomed well. In some embodiments of any of the methods described herein, each of the plurality of wells is a round-bottomed well. In some embodiments of any of the methods described herein, each of the plurality of wells has a perimeter that is hexagonally-shaped. In some embodiments of any of the methods described herein, each of the plurality of wells has a perimeter that is heptagonally-shaped. In some embodiments of any of the methods described herein, each of the plurality of wells has a perimeter that is octagonally-shaped. In some embodiments of any of the methods described herein, each of the plurality of wells has a perimeter that is pentagonally-shaped. In some embodiments of any of the methods described herein, each of the plurality of wells has a perimeter that is square-shaped. In some embodiments of any of the methods described herein, each of the plurality of wells has a perimeter that is circularly-shaped. In some embodiments of any of the methods described herein, each of the plurality of wells has substantially the same perimeter length. In some embodiments of any of the methods described herein, each of the plurality of wells do not have substantially the same perimeter length.

In some embodiments of any of the methods described herein, each of the plurality of wells has an opening and/or a bottom surface having an average diameter of about 3 μm to about 7 μm. In some embodiments of any of the methods described herein, each of the plurality of wells has an opening and/or a bottom surface having an average diameter of about 4 μm to about 6 μm. In some embodiments of any of the methods described herein, each of the plurality of wells has an opening and/or a bottom surface having an area of about 12 μm² to about 30 μm². In some embodiments of any of the methods described herein, each of the plurality of wells has an opening and/or a bottom surface having an area of about 15 μm² to about 27 μm². In some embodiments of any of the methods described herein, each of the plurality of wells has an opening and/or a bottom surface having an area of about 18 μm² to about 24 μm². In some embodiments of any of the methods described herein, each of the plurality of wells has a depth of about 10 μm to about 35 μm. In some embodiments of any of the methods described herein, each of the plurality of wells has a depth of about 15 μM to about 30 μm. In some embodiments of any of the methods described herein, each of the plurality of wells has a depth of about 20 μm to about 25 μm.

In some embodiments of any of the methods described herein, the plurality of wells has a geometric center-to-center spacing between adjacent wells of about 7.0 μm to about 20 μm. In some embodiments of any of the methods described herein, the plurality of wells has a geometric center-to-center spacing between adjacent wells of about 7.0 μm to about 10 μm. In some embodiments of any of the methods described herein, the plurality of wells has a geometric center-to-center spacing between adjacent wells of about 7.0 μm to about 8.5 μm.

In some embodiments of any of the methods described herein, the plurality of capture probes is disposed on a bottom surface of the well. In some embodiments of any of the methods described herein, the plurality of capture probes is disposed on one or more side surface(s) of the well. In some embodiments of any of the methods described herein, the plurality of capture probes is disposed on a bottom surface of the well and on one or more side surface(s) of the well.

In some embodiments of any of the methods described herein, the disposing in step (a) is performed using pressure applied using a roller or a stamping device. In some embodiments of any of the methods described herein, the plurality of probes were previously attached to a surface of the well using oligonucleotide photolithography. In some embodiments of any of the methods described herein, the plurality of probes were previously attached to a surface of the well using bridge amplification. In some embodiments of any of the methods described herein, the plurality of capture probes were previously disposed into the well by disposing a plurality of dissolvable hydrogel beads into the well, where the plurality of dissolvable hydrogels beads comprises the plurality of capture probes.

In some embodiments of any of the methods described herein, the releasing in step (c) comprises lysing the portion of the biological sample. Some embodiments of any of the methods described herein further include, prior to step (c), adding one or more lysis agent(s) to each of the plurality of wells. In some embodiments of any of the methods described herein, each of the plurality of wells in step (a) comprises one or more lysis agent(s).

Some embodiments of any of the methods described herein further include, prior to step (b), one or more of fixing, staining, and imaging the biological sample. In some embodiments of any of the methods described herein, the biological sample, prior to step (b), is disposed on a transparent substrate.

In some embodiments of any of the methods described herein, the biological sample is a tissue sample. In some embodiments of any of the methods described herein, the tissue sample is a fresh, frozen tissue sample. In some embodiments of any of the methods described herein, the tissue sample is a fixed tissue sample. In some embodiments of any of the methods described herein, the tissue sample is a formalin-fixed paraffin-embedded (FFPE) tissue sample.

In some embodiments of any of the methods described herein, step (d) comprises extending an end of the capture probe using the analyte specifically bound to the capture domain as a template. In some embodiments of any of the methods described herein, step (d) comprises sequencing (i) the sequence corresponding to the analyte or the complement thereof, and (ii) the sequence corresponding to the spatial barcode or the complement thereof. In some embodiments of any of the methods described herein, the sequencing is high throughput sequencing.

In some embodiments of any of the methods described herein, the analyte is RNA. In some embodiments of any of the methods described herein, the RNA is mRNA. In some embodiments of any of the methods described herein, the analyte is DNA. In some embodiments of any of the methods described herein, the DNA is genomic DNA.

Also provided herein are methods of determining a location of an analyte in a biological sample that include: (a) a substrate comprising a plurality of wells, where a well of the plurality of wells comprises: (i) a surface comprising a plurality of capture probes, where a capture probe of the plurality comprises a spatial barcode and a capture domain; and (ii) a plurality of analyte capture agents, where an analyte of the plurality of analyte capture agents comprises an analyte binding moiety, an analyte binding moiety barcode, and an analyte capture sequence; (b) disposing the biological sample into the plurality of wells; (c) releasing the analyte from the biological sample, where the analyte is specifically bound by the analyte binding moiety of the analyte capture agent, and the analyte capture sequence of the analyte capture agent is specifically bound by the capture domain of the capture probe; and (d) determining (i) a sequence corresponding to the analyte binding moiety barcode or a complement thereof, and (ii) a sequence corresponding to the spatial barcode or a complement thereof, and using the sequences of (i) and (ii) to determine the location of the analyte in the biological sample.

In some embodiments of any of the methods described herein, the substrate comprises about 1 million wells to about 5 million wells. In some embodiments of any of the methods described herein, each of the plurality of wells is a flat-bottomed well. In some embodiments of any of the methods described herein, each of the plurality of wells is a round-bottomed well. In some embodiments of any of the methods described herein, each of the plurality of wells has a perimeter that is hexagonally-shaped. In some embodiments of any of the methods described herein, each of the plurality of wells has a perimeter that is heptagonally-shaped. In some embodiments of any of the methods described herein, each of the plurality of wells has a perimeter that is octagonally-shaped. In some embodiments of any of the methods described herein, each of the plurality of wells has a perimeter that is pentagonally-shaped. In some embodiments of any of the methods described herein, each of the plurality of wells has a perimeter that is square-shaped. In some embodiments of any of the methods described herein, each of the plurality of wells has a perimeter that is circularly-shaped. In some embodiments of any of the methods described herein, each of the plurality of wells has substantially the same perimeter length. In some embodiments of any of the methods described herein, each of the plurality of wells do not have substantially the same perimeter length.

In some embodiments of any of the methods described herein, each of the plurality of wells has an opening and/or a bottom surface having an average diameter of about 3 μm to about 7 μm. In some embodiments of any of the methods described herein, each of the plurality of wells has an opening and/or a bottom surface having an average diameter of about 4 μm to about 6 μm. In some embodiments of any of the methods described herein, each of the plurality of wells has an opening and/or a bottom surface having an area of about 12 μm² to about 30 μm². In some embodiments of any of the methods described herein, each of the plurality of wells has an opening and/or a bottom surface having an area of about 15 μm² to about 27 μm². In some embodiments of any of the methods described herein, each of the plurality of wells has an opening and/or a bottom surface having an area of about 18 μm² to about 24 μm². In some embodiments of any of the methods described herein, each of the plurality of wells has a depth of about 10 μm to about 35 μm. In some embodiments of any of the methods described herein, each of the plurality of wells has a depth of about 15 μM to about 30 μm. In some embodiments of any of the methods described herein, each of the plurality of wells has a depth of about 20 μm to about 25 μm.

In some embodiments of any of the methods described herein, the plurality of wells has a geometric center-to-center spacing between adjacent wells of about 7.0 μm to about 20 μm. In some embodiments of any of the methods described herein, the plurality of wells has a geometric center-to-center spacing between adjacent wells of about 7.0 μm to about 10 μm. In some embodiments of any of the methods described herein, the plurality of wells has a geometric center-to-center spacing between adjacent wells of about 7.0 μm to about 8.5 μm.

In some embodiments of any of the methods described herein, the plurality of capture probes is disposed on a bottom surface of the well. In some embodiments of any of the methods described herein, the plurality of capture probes is disposed on one or more side surface(s) of the well. In some embodiments of any of the methods described herein, the plurality of capture probes is disposed on a bottom surface of the well and on one or more side surface(s) of the well.

In some embodiments of any of the methods described herein, the pressing in step (b) is performed using pressure applied using a roller or a stamping device. In some embodiments of any of the methods described herein, the plurality of probes were previously attached to a surface of the well using oligonucleotide photolithography. In some embodiments of any of the methods described herein, the plurality of probes were previously attached to a surface of the well using bridge amplification. In some embodiments of any of the methods described herein, the plurality of capture probes were previously disposed into the well by disposing a plurality of dissolvable hydrogel beads into the well, wherein the plurality of dissolvable hydrogels beads comprises the plurality of capture probes.

In some embodiments of any of the methods described herein, the releasing in step (c) comprises lysing the portion of the biological sample. Some embodiments of any of the methods described herein further include, prior to step (c), adding one or more lysis agent(s) to each of the plurality of wells. In some embodiments of any of the methods described herein, each of the plurality of wells in step (a) comprises one or more lysis agent(s).

Some embodiments of any of the methods described herein further include, prior to step (b), one or more of fixing, staining, and imaging the biological sample. In some embodiments of any of the methods described herein, the biological sample, prior to step (b), is disposed on a transparent substrate.

In some embodiments of any of the methods described herein, the biological sample is a tissue sample. In some embodiments of any of the methods described herein, the tissue sample is a fresh, frozen tissue sample. In some embodiments of any of the methods described herein, the tissue sample is a fixed tissue sample. In some embodiments of any of the methods described herein, the tissue sample is a formalin-fixed paraffin-embedded (FFPE) tissue sample.

In some embodiments of any of the methods described herein, step (d) comprises extending an end of the capture probe using the analyte binding moiety barcode as a template. In some embodiments of any of the methods described herein, step (d) comprises sequencing (i) the sequence corresponding to the analyte binding moiety barcode or the complement thereof, and (ii) the sequence corresponding to the spatial barcode or the complement thereof. In some embodiments of any of the methods described herein, the sequencing is high throughput sequencing.

In some embodiments of any of the methods described herein, the analyte binding moiety is an antibody or an antigen-binding fragment thereof. In some embodiments of any of the methods described herein, the analyte is a protein. In some embodiments of any of the methods described herein, the protein is an intracellular protein. In some embodiments of any of the methods described herein, the protein is an extracellular protein.

Also provided herein are devices that include a plurality of wells, where a well of the plurality of wells comprises a surface comprising a plurality of capture probes, where a capture probe of the plurality comprises a spatial barcode and a capture domain, and where: the device comprises about 1 million wells to about 5 million wells; each of the plurality of wells has an opening and/or a bottom surface having an average diameter of about 3 μm to about 7 μm; each of the plurality of wells has an opening and/or a bottom surface having an area of about 12 μm² to about 30 μm²; each of the plurality of wells has a depth of about 10 μm to about 35 μm; and the plurality of wells has a geometric center-to-center spacing between adjacent wells of about 7.0 μm to about 20 μm.

In some embodiments of any of the devices described herein, each of the plurality of wells is a flat-bottomed well. In some embodiments of any of the devices described herein, each of the plurality of wells is a round-bottomed well.

In some embodiments of any of the devices described herein, each of the plurality of wells has a perimeter that is hexagonally-shaped, heptagonally-shaped, octagonally-shaped, pentagonally-shaped, square-shaped, or circularly-shaped. In some embodiments of any of the devices described herein, each of the plurality of wells has substantially the same perimeter length. In some embodiments of any of the devices described herein, each of the plurality of wells do not have substantially the same perimeter length.

In some embodiments of any of the devices described herein, each of the plurality of wells has an opening and/or a bottom surface having an average diameter of about 4 μm to about 6 μm. In some embodiments of any of the devices described herein, each of the plurality of wells has an opening and/or a bottom surface having an area of about 15 μm² to about 27 μm². In some embodiments of any of the devices described herein, each of the plurality of wells has an opening and/or a bottom surface having an area of about 18 μm² to about 24 μm². In some embodiments of any of the devices described herein, each of the plurality of wells has a depth of about 15 μm to about 30 μm. In some embodiments of any of the devices described herein, each of the plurality of wells has a depth of about 20 μm to about 25 μm. In some embodiments of any of the devices described herein, the plurality of wells has a geometric center-to-center spacing between adjacent wells of about 7.0 μm to about 10 μm. In some embodiments of any of the devices described herein, the plurality of wells has a geometric center-to-center spacing between adjacent wells of about 7.0 μm to about 8.5 μm.

In some embodiments of any of the devices described herein, the plurality of capture probes is disposed on a bottom surface of the well. In some embodiments of any of the devices described herein, the plurality of capture probes is disposed on one or more side surface(s) of the well. In some embodiments of any of the devices described herein, the plurality of capture probes is disposed on a bottom surface of the well and on one or more side surface(s) of the well. In some embodiments of any of the devices described herein, the plurality of probes were previously attached to a surface of the well using oligonucleotide photolithography. In some embodiments of any of the devices described herein, the plurality of probes were previously attached to a surface of the well using bridge amplification.

In some embodiments of any of the devices described herein, the plurality of capture probes were previously disposed into the well by disposing a plurality of dissolvable hydrogel beads into the well, where the plurality of dissolvable hydrogels beads comprises the plurality of capture probes.

In some embodiments of any of the devices described herein, each of the plurality of wells further comprises one or more lysis agent(s). In some embodiments of any of the devices described herein, each of the plurality of wells further comprises one or more analyte capture agents.

Also provided herein are devices that include a plurality of wells, where a well of the plurality of wells comprises a surface comprising a plurality of capture probes, where a capture probe of the plurality comprises in a 5′ to 3′ direction, a spatial barcode, and a capture domain, and a sequence complementary to at least a portion of a sequence of an analyte from a biological sample, and where: the substrate device comprises about 1 million wells to about 5 million wells; each of the plurality of wells has an opening and/or a bottom surface having an average diameter of about 3 μm to about 7 μm; each of the plurality of wells has an opening and/or a bottom surface having an area of about 12 μm² to about 30 μm²; each of the plurality of wells has a depth of about 10 μm to about 35 μm; and the plurality of wells has a geometric center-to-center spacing between adjacent wells of about 7.0 μm to about 20 μm.

In some embodiments of any of the devices described herein, each of the plurality of wells is a flat-bottomed well. In some embodiments of any of the devices described herein, each of the plurality of wells is a round-bottomed well. In some embodiments of any of the devices described herein, each of the plurality of wells has a perimeter that is hexagonally-shaped, heptagonally-shaped, octagonally-shaped, pentagonally-shaped, square-shaped, or circularly-shaped. In some embodiments of any of the devices described herein, each of the plurality of wells has substantially the same perimeter length. In some embodiments of any of the devices described herein, each of the plurality of wells do not have substantially the same perimeter length.

In some embodiments of any of the devices described herein, each of the plurality of wells has an opening and/or a bottom surface having an average diameter of about 4 μm to about 6 μm. In some embodiments of any of the devices described herein, each of the plurality of wells has an opening and/or a bottom surface having an area of about 15 μm² to about 27 μm². In some embodiments of any of the devices described herein, each of the plurality of wells has an opening and/or a bottom surface having an area of about 18 μm² to about 24 μm². In some embodiments of any of the devices described herein, each of the plurality of wells has a depth of about 15 μm to about 30 μm. In some embodiments of any of the devices described herein, each of the plurality of wells has a depth of about 20 μm to about 25 μm. In some embodiments of any of the devices described herein, the plurality of wells has a geometric center-to-center spacing between adjacent wells of about 7.0 μm to about 10 μm. In some embodiments of any of the devices described herein, the plurality of wells has a geometric center-to-center spacing between adjacent wells of about 7.0 μm to about 8.5 μm.

In some embodiments of any of the devices described herein, the plurality of capture probes is disposed on a bottom surface of the well. In some embodiments of any of the devices described herein, the plurality of capture probes is disposed on one or more side surface(s) of the well. In some embodiments of any of the devices described herein, the plurality of capture probes is disposed on a bottom surface of the well and on one or more side surface(s) of the well. In some embodiments of any of the devices described herein, the plurality of probes were previously attached to a surface of the well using oligonucleotide photolithography. In some embodiments of any of the devices described herein, the plurality of probes were previously attached to a surface of the well using bridge amplification. In some embodiments of any of the devices described herein, the plurality of capture probes were previously disposed into the well by disposing a plurality of dissolvable hydrogel beads into the well, wherein the plurality of dissolvable hydrogels beads comprises the plurality of capture probes.

In some embodiments of any of the devices described herein, each of the plurality of wells further comprises one or more lysis agent(s).

In some embodiments of any of the devices described herein, each of the plurality of wells further comprises one or more analyte capture agents.

Also provided herein are kits that include any of the devices described herein. Some embodiments of any of the kits described herein further include instructions for performing any of the methods described herein. Some embodiments of any of the kits described herein further include one or more of a reverse transcriptase, a polymerase, an RNase, a protease, a DNase, and a lipase. Some embodiments of any of the kits described herein further include a lysis agent.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows an exemplary spatial analysis workflow.

FIG. 2 shows an exemplary spatial analysis workflow.

FIG. 3 is a schematic diagram showing an example of a barcoded capture probe, as described herein.

FIG. 4 is a schematic illustrating a cleavable capture probe, wherein the cleaved capture probe can enter into a non-permeabilized cell and bind to target analytes within the sample.

FIG. 5 is a schematic diagram of an exemplary multiplexed spatially-barcoded feature.

FIG. 6A is a workflow schematic illustrating exemplary, non-limiting, non-exhaustive steps for “pixelating” a sample, wherein the sample is cut, stamped, microdissected, or transferred by hollow-needle or microneedle, moving a small portion of the sample into an individual partition or well.

FIG. 6B is a schematic depicting multi-needle pixilation, wherein an array of needles is punched through a sample on a scaffold into nanowells containing gel beads and reagents. Once the needle is in the nanowell, the cell(s) are ejected.

FIGS. 7A-7B show 7A) a side-view of an example well array with unique spatially-barcoded capture probes in each well and 7B) an oblique top-view of an example well array.

FIGS. 8A-8B show 8A) an example tissue section and the relative positioning of the tissue section above the spatially-barcoded well array, and 8B) the spatially-barcoded well array after the tissue section has been pressed into the wells resulting in sectioning of the cells of the tissue section into the wells.

FIG. 9A-9C show 9A) an example means of placing the spatially-barcoded capture probes within the wells using photolithography, 9B) an example means of placing the spatially-barcoded capture probes within the wells using barcoded microbeads, and 9C) an example means of placing the spatially-barcoded capture probes within the wells using bridge amplification.

FIGS. 10A-10J show 10A) an exemplary bridge amplification method including a first primer sequence and a second primer sequence, and a first adapter sequence, 10B) the first exemplary primer sequence is extended to form a first complementary strand in the first step of a bridge amplification, 10C) the first complementary strand is bound to the second primer sequence in the second step of bridge amplification, 10D) the second primer is extended to form a first reverse sequence complementary strand in the third step of bridge amplification, 10E) the first complementary strand and first reverse sequence complementary strand, and a third and fourth primer sequence in the fourth step of bridge amplification, 10F) the result of repeating the second, third, and fourth steps of bridge amplification resulting in a second complementary strand and second reverse sequence complementary strand, 10G) the end result of bridge amplification following the removal of reverse-sequence complementary strands, 10H) the first complementary strand bound to an adapter sequence, 10I) the first complementary strand is extended using the adapter sequence as a template to form a capture probe, and 10J) a completed exemplary capture probe as the result of the bridge amplification.

FIGS. 11A-11B show 11A) an example tissue section and the relative positioning of the tissue section above the spatially-barcoded well array, and 11B) the spatially-barcoded well array after the tissue section has been contacted to the upper surface of the wells and electrodes placed on opposing sides of the spatially-barcoded well array and tissue section.

FIGS. 12A-12B show 12A) an example tissue section and a spatially-barcoded well array after the tissue section has been pressed into the wells resulting in sectioning of the cells of the tissue section into the wells, and 12B) the spatially-barcoded well array after the tissue section has been pressed into the wells and electrodes placed on opposing sides of the spatially-barcoded well array and tissue section.

FIG. 13A-13B show 13A) a microwell array and stage with an XY microcontroller, and 13B) a tissue sample being positioned above the microwell array by the stage and XY microcontroller.

### DETAILED DESCRIPTION

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/71796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2021/102039 and/or U.S. patent application Ser. No. 16/951,864, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of each analyte within the biological sample. The spatial location of each analyte within the biological sample is determined based on the feature to which each analyte is bound on the array, and the feature's relative spatial location within the array.

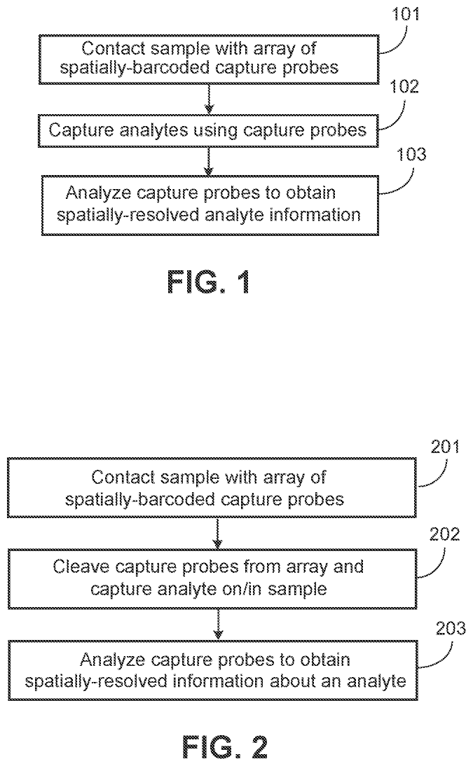

There are at least two general methods to associate a spatial barcode with one or more neighboring cells, such that the spatial barcode identifies the one or more cells, and/or contents of the one or more cells, as associated with a particular spatial location. One general method is to promote analytes out of a cell and towards the spatially-barcoded array. FIG. 1 depicts an exemplary embodiment of this general method. In FIG. 1, the spatially-barcoded array populated with capture probes (as described further herein) is contacted with a biological sample 101, and biological sample is permeabilized, allowing the analyte to migrate away from the sample and toward the array. The analyte interacts with a capture probe on the spatially-barcoded array 102. Once the analyte hybridizes/is bound to the capture probe, the sample is optionally removed from the array and the capture probes are analyzed in order to obtain spatially-resolved analyte information 103.

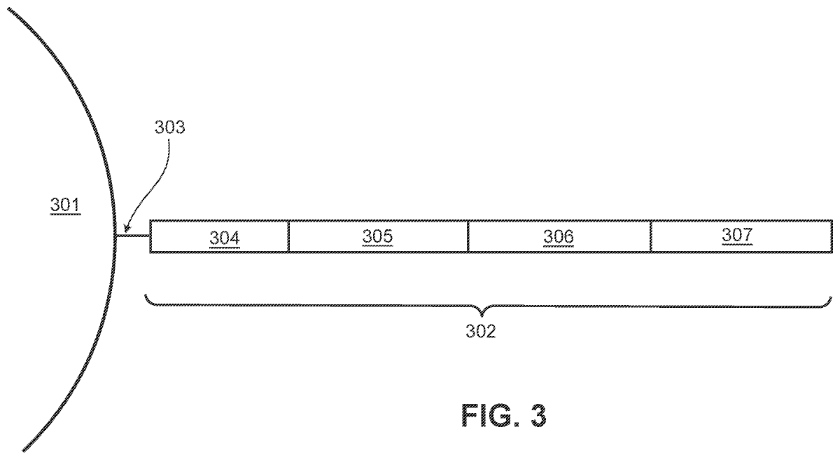

Another general method is to cleave the spatially-barcoded capture probes from an array, and promote the spatially-barcoded capture probes towards and/or into or onto the biological sample. FIG. 2 depicts an exemplary embodiment of this general method, the spatially-barcoded array populated with capture probes (as described further herein) can be contacted with a sample 201. The spatially-barcoded capture probes are cleaved and then interact with cells within the provided biological sample 202. The interaction can be a covalent or non-covalent cell-surface interaction. The interaction can be an intracellular interaction facilitated by a delivery system or a cell penetration peptide. Once the spatially-barcoded capture probe is associated with a particular cell, the sample can be optionally removed for analysis. The sample can be optionally dissociated before analysis. Once the tagged cell is associated with the spatially-barcoded capture probe, the capture probes can be analyzed to obtain spatially-resolved information about the tagged cell 203. A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe is a conjugate (e.g., an oligonucleotide-antibody conjugate). In some embodiments, the capture probe includes a barcode (e.g., a spatial barcode and/or a unique molecular identifier (UMI)) and a capture domain.

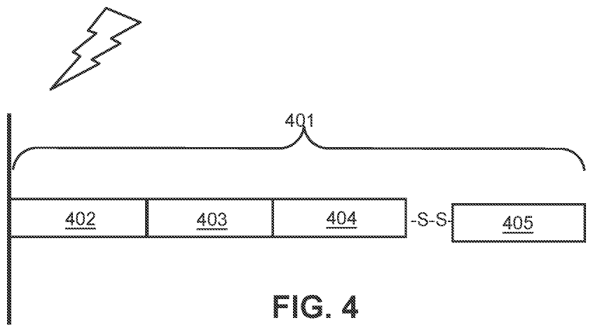

FIG. 3 is a schematic diagram showing an example of a capture probe, as described herein. As shown, the capture probe 302 is optionally coupled to a feature 301 by a cleavage domain 303, such as a disulfide linker. The capture probe can include functional sequences that are useful for subsequent processing, such as functional sequence 304, which can include a sequencer specific flow cell attachment sequence, e.g., a P5 or P7 sequence, as well as functional sequence 306, which can include sequencing primer sequences, e.g., a R1 primer binding site, a R2 primer binding site. In some embodiments, sequence 304 is a P7 sequence and sequence 306 is a R2 primer binding site. A spatial barcode 305 can be included within the capture probe for use in barcoding the target analyte. The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 305, functional sequences 304 (e.g., flow cell attachment sequence) and 306 (e.g., sequencing primer sequences) can be common to all of the probes attached to a given feature. The spatial barcode can also include a capture domain 307 to facilitate capture of a target analyte.

Each capture probe can optionally include at least one cleavage domain. The cleavage domain represents the portion of the probe that is used to reversibly attach the probe to an array feature, as will be described further herein. Further, one or more segments or regions of the capture probe can optionally be released from the array feature by cleavage of the cleavage domain. As an example, spatial barcodes and/or universal molecular identifiers (UMIs) can be released by cleavage of the cleavage domain.

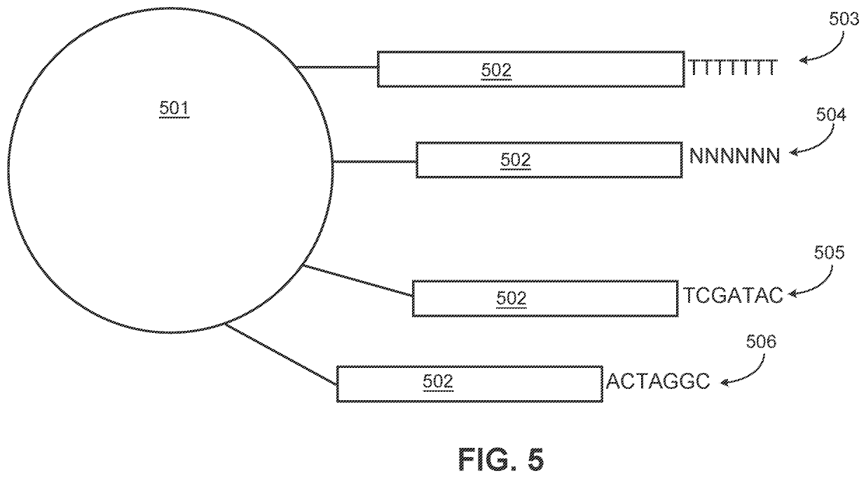

FIG. 4 is a schematic illustrating a cleavable capture probe, wherein the cleaved capture probe can enter into a non-permeabilized cell and bind to analytes within the sample. The capture probe 401 contains a cleavage domain 402, a cell penetrating peptide 403, a reporter molecule 404, and a disulfide bond (—S—S—). 405 represents all other parts of a capture probe, for example a spatial barcode and a capture domain.

For multiple capture probes that are attached to a common array feature, the one or more spatial barcode sequences of the multiple capture probes can include sequences that are the same for all capture probes coupled to the feature, and/or sequences that are different across all capture probes coupled to the feature.

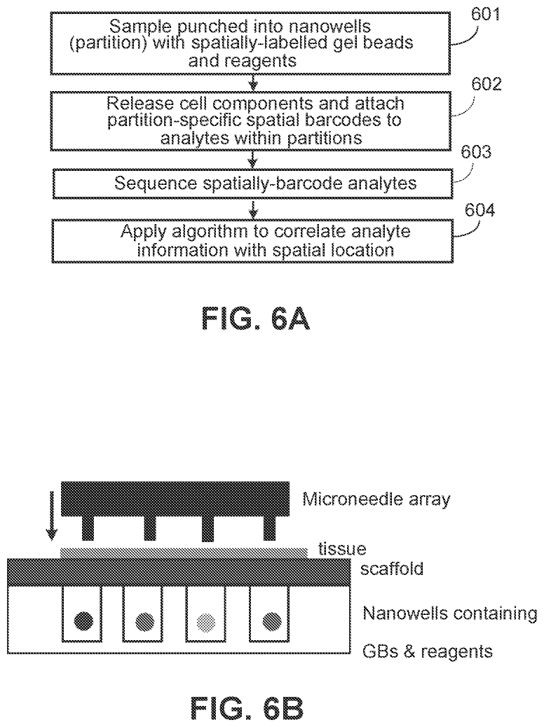

FIG. 5 is a schematic diagram of an exemplary multiplexed spatially-barcoded feature. In FIG. 5, the feature 501 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 502. One type of capture probe associated with the feature includes the spatial barcode 502 in combination with a poly(T) capture domain 503, designed to capture mRNA target analytes. A second type of capture probe associated with the feature includes the spatial barcode 502 in combination with a random N-mer capture domain 504 for gDNA analysis. A third type of capture probe associated with the feature includes the spatial barcode 502 in combination with a capture domain complementary to the analyte capture agent of interest 505. A fourth type of capture probe associated with the feature includes the spatial barcode 502 in combination with a capture probe that can specifically bind a nucleic acid molecule 506 that can function in a CRISPR assay (e.g., CRISPR/Cas9). While only four different capture probe-barcoded constructs are shown in FIG. 5, capture-probe barcoded constructs can be tailored for analyses of any given analyte associated with a nucleic acid and capable of binding with such a construct. For example, the schemes shown in FIG. 5 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

Some embodiments of any of the methods described herein can include separating a biological sample into single cells, cell groups, types of cells, or a region or regions of interest. For example, a biological sample can be separated into single cells, cell groups, types of cells, or a region or regions of interest before being contacted with one or more capture probes. In other examples, a biological sample is first contacted with one or more capture probes, and then separated into single cells, cell groups, types of cells, or a region or regions of interest.

In some embodiments, a biological sample can be separated into chucks using pixelation. Pixelation can include the steps of providing a biological sample, and punching out one or more portions of the biological sample. The punched out portions of the biological sample can then be used to perform any of the methods described herein. In some embodiments, the punched-out portions of the biological sample can be in a random pattern or a designed pattern. In some embodiments, the punched-out portions of the biological sample can be focused on a region of interest or a subcellular structure in the biological sample.

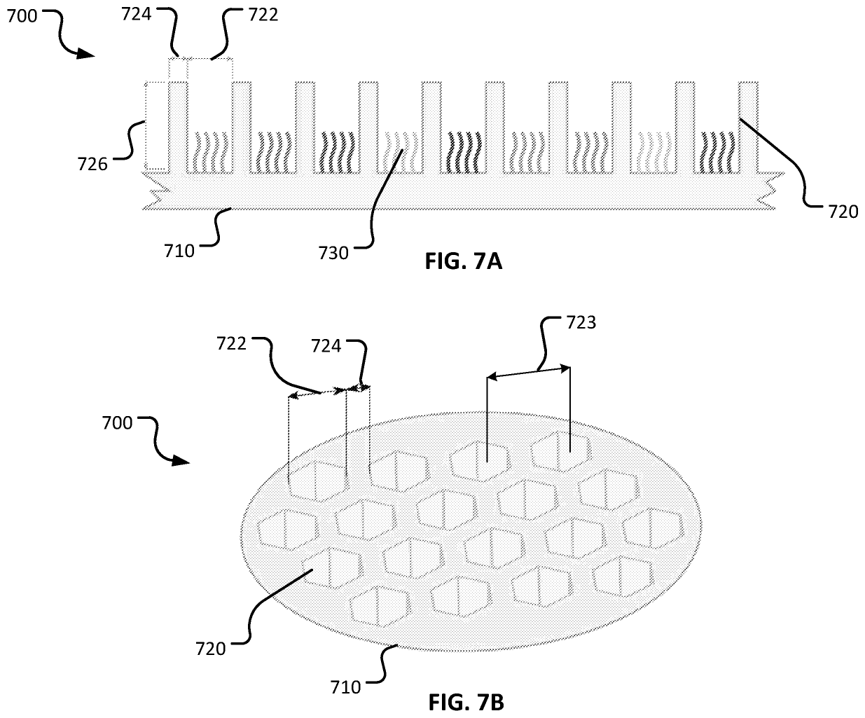

FIG. 6A is a workflow schematic illustrating exemplary, non-limiting, non-exhaustive steps for “pixelating” a sample, wherein the sample is cut, stamped, microdissected, or transferred by hollow-needle or microneedle, moving a small portion of the sample into an individual partition or well.

FIG. 6B is a schematic depicting multi-needle pixelation, wherein an array of needles is punched through a sample on a scaffold, into nanowells containing beads (e.g., gel beads) and reagents. Once the needle is in the nanowell, the cell(s) are ejected.

### I. Identifying a Location of an Analyte in a Biological Sample

The ability to spatially determine the location of a target analyte in a biological sample such as a cell or tissue, is a powerful tool in the cellular toolbox for scientific research. Loss of spatial information due to diffusion of target analytes within and surrounding a cell or tissue can result in loss of sensitivity and decreased spatial resolution, for example when trying to capture the analytes that corresponds to where they occur natively within a cell or tissue. This disclosure provides solutions for issues resulting from diffusion or other mechanisms that may displace target analytes outside what would correspond to their native context within a cell or tissue. By providing a microenvironment for cells or subsets of tissues (e.g, two or more cells), diffusion or dispersal of the target analytes prior to capture can be mitigated or eliminated, thereby allowing for greater sensitivity and spatial resolution.

(a) Methods for Identifying a Location of an Analyte in a Biological Sample

Disclosed herein are methods of using a substrate containing a plurality of wells and spatially-barcoded arrays affixed within the wells to determine a location of an analyte in a biological sample (e.g., any of the exemplary biological samples described herein) that include (a) disposing portions of a biological sample into a plurality of wells, where a well of the plurality of wells comprises a surface comprising a plurality of capture probes, where a capture probe of the plurality comprises a spatial barcode and a capture domain; (b) releasing the analyte from the biological sample, where the analyte is specifically bound by the capture domain of the capture probe; and (c) determining (i) a sequence corresponding to the analyte or a complement thereof, and (ii) a sequence corresponding to the spatial barcode or a complement thereof, and using the sequences of (i) and (ii) to determine the location of the analyte in the biological sample. The methods are particularly advantageous, as the disposal of a portion of a biological sample into a well of the plurality of wells allows for the lysis of the portion of the biological sample in the well, and analysis of analytes from the cell(s) within the well. For example, lysis of a portion of the biological sample (e.g., a cell, two cells, three cells, etc.) can occur within a well of the plurality of wells. The methods also provide a reduction in the volume of reagents needed to perform the methods described herein. The methods also provide for a reduction in diffusion of analytes during analysis. The methods provided herein also include the use of wells that have capture probes on the sides of the well, which can provide for three-dimensional analysis of analytes from the portion of the biological sample in a well.

In some embodiments, the substrate can include a plurality of wells. In some embodiments, the plurality of wells are constrained to an area of the substrate. In some embodiments, the area of the substrate that includes the plurality of wells can be about 6.5 mm by about 6.5 mm (e.g., about 6.1 mm by about 6.5 mm, about 6.2 mm by about 6.5 mm, about 6.3 mm by about 6.5 mm, about 6.4 mm by about 6.5 mm, about 6.6 mm by about 6.5 mm, about 6.7 mm by about 6.5 mm, about 6.8 mm by about 6.5 mm, about 6.9 mm by about 6.5 mm, about 6.5 mm by about 6.1 mm, about 6.5 mm by about 6.2 mm, about 6.5 mm by about 6.3 mm, about 6.5 mm by about 6.4 mm, about 6.5 mm by about 6.6 mm, about 6.5 mm by about 6.7 mm, about 6.5 mm by about 6.8 mm, or about 6.5 mm by about 6.9 mm).

In some embodiments, the area of the substrate that includes the plurality of wells can be about 11 mm by about 11 mm (e.g., about 11.1 mm by about 11 mm, about 11.2 mm by about 11 mm, about 11.3 mm by about 11 mm, about 11.4 mm by about 11 mm, about 11.6 mm by about 11 mm, about 11.7 mm by about 11 mm, about 11.8 mm by about 11 mm, about 11.9 mm by about 11 mm, about 11 mm by about 11.1 mm, about 11 mm by about 11.2 mm, about 11 mm by about 11.3 mm, about 11 mm by about 11.4 mm, about 11 mm by about 11.6 mm, about 11 mm by about 11.7 mm, about 11 mm by about 11.8 mm, or about 11 mm by about 11.9 mm).

In some embodiments, the area of the substrate can include about 1 million to about 5 million wells (e.g., about 1.4 million to about 5 million, about 1.8 million to about 5 million, about 2.2 million to about 5 million, about 2.6 million to about 5 million, about 3 million to about 5 million, about 3.4 million to about 5 million, about 3.8 million to about 5 million, about 4.2 million to about 5 million, about 4.6 million to about 5 million, about 1 million to about 4.6 million, about 1 million to about 4.2 million, about 1 million to about 3.8 million, about 1 million to about 3.4 million, about 1 million to about 3 million, about 1 million to about 2.6 million, about 1 million to about 2.2 million, about 1 million to about 1.8 million, or about 1 million to about 1.4 million).

In some embodiments, the plurality of wells can be arranged on a substrate in a regular fashion. In some embodiments, the plurality of wells can be arranged on a substrate in an irregular fashion.

In some embodiments, the opening of each well of the plurality of wells can have a geometric shape. For example, the opening of a well can have a circular, triangular, square, pentagonal, hexagonal, heptagonal, or octagonal shape. In some embodiments, the opening of a well can have a number of sides. For example, the opening of each well of the plurality of wells can have between 3 and 10 sides (e.g., between 4 and 10, between 5 and 10, between 6 and 10, between 7 and 10, between 8 and 10, between 9 and 10, between 3 and 9, between 3 and 8, between 3 and 7, between 3 and 6, between 3 and 5, or between 3 and 4 sides). In some embodiments, the opening of each well of the plurality of wells can be substantially the same for all the wells of the plurality of wells. In some embodiments, the opening of each well of the plurality of wells can be substantially different for all the wells of the plurality of wells.

In some embodiments, the opening of each well of the plurality of wells can have a diameter between about 3 μm and about 7 μm (e.g., between about 3.4 μm and about 7 μm, between about 3.8 μm and about 7 μm, between about 4.2 μm and about 7 μm, between about 4.6 μm and about 7 μm, between about 5 μm and about 7 μm, between about 5.4 μm and about 7 μm, between about 5.8 μm and about 7 μm, between about 6.2 μm and about 7 μm, between about 6.6 μm and about 7 μm, between about 3 μm and about 6.6 μm, between about 3 μm and about 6.2 μm, between about 3 μm and about 5.8 μm, between about 3 μm and about 5.4 μm, between about 3 μm and about 5 μm, between about 3 μm and about 4.6 μm, between about 3 μm and about 4.2 μm, between about 3 μm and about 3.8 μm, or between about 3 μm and about 3.4 μm).

In some embodiments, the opening of each well of the plurality of wells can have an area in the range between about 15 μm² to about 27 μm² (e.g., about 17 μm² to about 27 μm², about 18 μm² to about 27 μm², about 19 μm² to about 27 μm², about 20 μm² to about 27 μm², about 21 μm² to about 27 μm², about 22 μm² to about 27 μm², about 23 μm² to about 27 μm², about 24 μm² to about 27 μm², about 25 μm² to about 27 μm², about 26 μm² to about 27 μm², about 15 μm² to about 26 μm², about 15 μm² to about 25 μm², about 15 μm² to about 24 μm², about 15 μm² to about 23 μm², about 15 μm² to about 22 μm², about 15 μm² to about 21 μm², about 15 μm² to about 20 μm², about 15 μm² to about 19 μm², about 15 μm² to about 18 μm², about 15 μm² to about 17 μm², or about 15 μm² to about 16 μm²).

In some embodiments, the bottom of each well of the plurality of wells can have a surface area in the range between about 15 μm² to about 27 μm² (e.g., about 17 μm² to about 27 μm², about 18 μm² to about 27 μm², about 19 μm² to about 27 μm², about 20 μm² to about 27 μm², about 21 μm² to about 27 μm², about 22 μm² to about 27 μm², about 23 μm² to about 27 μm², about 24 μm² to about 27 μm², about 25 μm² to about 27 μm², about 26 μm² to about 27 μm², about 15 μm² to about 26 μm², about 15 μm² to about 25 μm², about 15 μm² to about 24 μm², about 15 μm² to about 23 μm², about 15 μm² to about 22 μm², about 15 μm² to about 21 μm², about 15 μm² to about 20 μm², about 15 μm² to about 19 μm², about 15 μm² to about 18 μm², about 15 μm² to about 17 μm², or about 15 μm² to about 16 μm²).

In some embodiments, an edge of an aperture of a well of the plurality of wells can be separated by from an edge of an aperture of an adjacent well by about 1 μm to about 3 μm (e.g., about 1.2 μm to about 3 μm, about 1.4 μm to about 3 μm, about 1.6 μm to about 3 μm, about 1.8 μm to about 3 μm, about 2 μm to about 3 μm, about 2.2 μm to about 3 μm, about 2.4 μm to about 3 μm, about 2.6 μm to about 3 μm, about 2.8 μm to about 3 μm, about 1 μm to about 2.8 μm, about 1 μm to about 2.6 μm, about 1 μm to about 2.4 μm, about 1 μm to about 2.2 μm, about 1 μm to about 2 μm, about 1 μm to about 1.8 μm, about 1 μm to about 1.6 μm, about 1 μm to about 1.4 μm, or about 1 μm to about 1.2 μm).

In some embodiments, each well of the plurality of wells can be separated by a geometric center-to-center distance of about 7.0 μm to about 25 μm (e.g., about 7.0 μm to about 20 μm, about 7.0 μm to about 15 μm, about 7.0 μm to about 12 μm, about 7.0 μm to about 10 μm, about 7.0 μm to about 8.5 μm, about 8.5 μm to about 25 μm, about 8.5 μm to about 20 μm, about 8.5 μm to about 15 μm, about 8.5 μm to about 12 μm, about 8.5 μm to about 10 μm, about 10 μm to about 25 μm, about 10 μm to about 15 μm, or about 15 μm to about 20 μm).

In some embodiments, the depth of each well of the plurality of wells can be between about 10 μm to about 35 μm (e.g., about 10 μm to about 32.5 μm, about 10 μm to about 30 μm, about 10 μm to about 27.5 μm, about 10 μm to about 25 μm, about 10 μm to about 22.5 μm, about 10 μm to about 20 μm, about 10 μm to about 17.5 μm, about 10 μm to about 15 μm, about 10 μm to about 12.5 μm, about 12.5 μm to about 35 μm, about 15 μm to about 35 μm, about 17.5 μm to about 35 μm, about 20 μm to about 35 μm, about 22.5 μm to about 35 μm, about 25 μm to about 35 μm, about 27.5 μm to about 35 μm, about 30 μm to about 35 μm, or about 32.5 μm to about 35 μm).

In some embodiments, each well of the plurality of wells can have a flat bottom. In some embodiments, each well of the plurality of wells can have a rounded (e.g., hemispherical) bottom. In some embodiments, a rounded bottom of a well can have either a convex or concave shape.

In some embodiments, a well comprises a plurality of capture probes. In some embodiments, the capture probes can include a spatial barcode. In some embodiments, the capture probes can include a capture domain (e.g., any of the exemplary capture domains described herein). In some embodiments, the capture probes can include a cleavage site. In some embodiments, the capture probes can be attached to a surface of a well or a bead disposed in a well. In some embodiments, the capture probe can include a functional sequence. In some embodiments, the functional sequence can be a unique molecular identifier (UMI) sequence. In some embodiments, the UMI can be positioned 5′ relative to the capture domain.

In some embodiments, the capture domain can be any agent that is capable of capturing a target analyte. In some embodiments, the capture domain can be located at the 3′ end of the capture probe. In some embodiments, the capture domain can be a homopolynucleotide sequence, e.g., a poly (dT) sequence, a poly (dA) sequence, a poly (dG) sequence, or a poly (dC) sequence. In some embodiments, the capture domain can be a gene-specific sequence (e.g., a sequence complementary to a specific target analyte).

In some embodiments, the plurality of capture probes can be affixed to a plurality of beads (e.g., any of the exemplary beads described herein). In some embodiments, the plurality of beads can be a plurality of hydrogel beads. In some embodiments, the hydrogel beads can be made of a natural material, synthetic material, or a combination thereof. In some embodiments, the hydrogel beads can be dissolvable hydrogel beads (e.g., conditionally dissolvable polymer, e.g., DTT sensitive hydrogel).

In general, each well can contain at least one bead. In some embodiments, each well can contain more than one bead (e.g., two beads, three beads, or four beads). In some embodiments, the bead can have a diameter of between about 3 μm to about 6 μm (e.g., about 3.5 μm to about 6 μm, about 4 μm to about 6 μm, about 4.5 μm to about 6 μm, about 5 μm to about 6 μm, about 5.5 μm to about 6 μm, about 3 μm to about 5.5 μm, about 3 μm to about 5 μm, about 3 μm to about 4.5 μm, about 3 μm to about 4 μm, or about 3 μm to about 3.5 μm).

In some embodiments, the plurality of capture probes can be affixed to one or more surfaces of each well. For example, the plurality of capture probes can be affixed to the bottom surface of each well. In some examples, the plurality of capture probes can be affixed to one or more side surface(s) of each well. In some embodiments, the plurality of capture probes can be affixed to at least one surface (e.g., two surfaces, three surfaces, four surfaces, five surfaces, six surfaces, seven surfaces, eight surfaces, nine surfaces, or ten surfaces). In some embodiments, the plurality of capture probes can be affixed to both the bottom and one or more sides of a well.

In some embodiments, the plurality of capture probes can be affixed to one or more surfaces of each well through lithographic methods. In some embodiments, the plurality of capture probes can be affixed to one or more surfaces of each well through photolithographic methods. In some embodiments, a linker can be affixed to one or more surfaces of each well through covalent binding to begin the photolithographic method.

In some embodiments, the capture probes can be constructed onto the linker through photolithographic methods. In some embodiments, the linker can be protected by a photolabile protecting group. In some embodiments, the protecting group can include nitrobenzyl protecting groups. In some embodiments, the protecting group can include benzyl protecting groups. In some embodiments, the protecting group can include carbonyl protecting groups.

In some embodiments, the nucleotide photolithography can include exposing the well array to light. In some embodiments, the light can be ultraviolet (UV) light. For example, wavelength of the light can be between about 10 nm and about 400 nm (e.g., about 50 nm to about 400 nm, about 100 nm to about 400 nm, about 150 nm to about 400 nm, about 200 nm to about 400 nm, about 250 nm to about 400 nm, about 300 nm to about 400 nm, about 350 nm to about 400 nm, about 10 nm to about 350 nm, about 10 nm to about 300 nm, about 10 nm to about 250 nm, about 10 nm to about 200 nm, about 10 nm to about 150 nm, about 10 nm to about 100 nm, about 10 nm to about 50 nm, about 100 nm to about 300 nm, about 200 nm to about 300 nm, or about 100 nm to about 200 nm). It should be understood that the wavelength of the light can be selected based on the protecting group used.

In some embodiments, the nucleotide photolithography can include exposing the well array to light through a mask. In some embodiments, the mask can protect at least one well from exposure to light. For example, the mask can protect at least 1% of the wells (e.g., at least 5% of the wells, at least 10% of the wells, at least 20% of the wells, at least 40% of the wells, at least 60% of the wells, at least 80% of the wells, at least 90% of the wells, at least 95% of the wells, or at least 99% of the wells).

In some embodiments, the well array can be exposed to UV light a plurality of times. In some embodiments, the well array can be exposed to UV light between 10 and 100 times (e.g., between 20 and 100 times, between 30 and 100 times, between 40 and 100 times, between 50 and 100 times, between 60 and 100 times, between 70 and 100 times, between 80 and 100 times, between 90 and 100 times, between 10 and 90 times, between 10 and 80 times, between 10 and 70 times, between 10 and 60 times, between 10 and 50 times, between 10 and 40 times, between 10 and 30 times, or between 10 and 20 times).

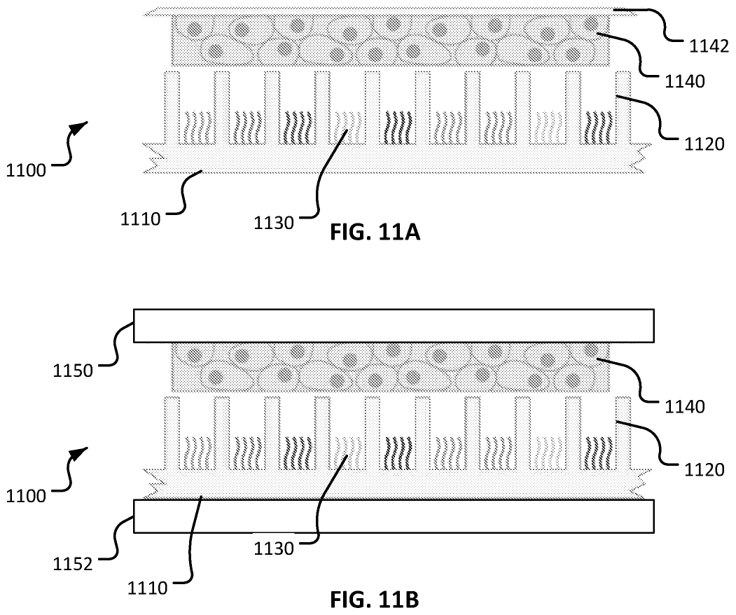

In some embodiments, the plurality of capture probes can be affixed to one or more surfaces of each well through amplification methods. In some embodiments, the plurality of capture probes can be affixed to one or more surfaces of each well through bridge amplification methods. As an example, primers are first attached to one or more surfaces of the plurality of wells. In some embodiments, two primers (e.g., a forward primer and a reverse primer) are first attached to one or more surfaces of the plurality of wells. The primers are then amplified so that local clonal colonies are formed (FIG. 10).

In the present disclosure, the biological sample is disposed into one or more wells of the plurality of wells. In some embodiments, pressure is applied to the biological sample to partition) the biological sample into the one or more wells of the plurality of wells. For example, pressure can be applied using a roller or a stamping device. In some embodiments, the pressure can be applied using a hand or finger of a user.

In some embodiments, the biological sample is a tissue sample. In some embodiments, the biological sample is a section of a tissue sample. In some embodiments, the biological sample is a fresh tissue sample. In some embodiments, the biological sample is a fresh, frozen tissue sample. In some embodiments, the biological sample is a fixed tissue sample, e.g., a formalin-fixed and paraffin-embedded (FFPE) sample. In some embodiments, the biological sample is a tissue sample embedded in optimal cutting temperature (OCT) compound.

The method further includes releasing one or more target analyte(s) from the biological sample, where a target analyte of the one or more target analyte(s) that is released from the biological sample is specifically bound, or hybridized, by the capture domain(s) of the capture probe(s) in a well.

In some embodiments, the biological sample is lysed to release the one or more target analyte(s). Lysis solutions can include ionic surfactants such as, for example, sarcosyl and sodium dodecyl sulfate (SDS). More generally, chemical lysis agents can include, without limitation, organic solvents, chelating agents, detergents (e.g., ionic, anionic, zwitterionic, etc.), surfactants, and chaotropic agents. Additional examples of lysis agents are described herein.

In some embodiments, the target analyte is a nucleic acid. In some embodiments, the nucleic acid is DNA (e.g., genomic DNA or mitochondrial DNA). In some embodiments, the nucleic acid is RNA. In some embodiments, the RNA includes any of the RNA molecules as described herein (e.g., mRNA). In some embodiments, the target analyte is a protein. In some embodiments, the protein is an intracellular protein, extracellular protein, or a cell surface protein.

(c) Kits

Also provided herein are kits that can be used to perform any of the methods described herein. For example, provided herein are kits that include any of the devices described herein. In some examples, a kit can further include instructions for performing any of the methods described herein. In some examples, a kit can further include one or more of a reverse transcriptase, a polymerase, an RNase, a protease, a DNase, and a lipase. In some examples, a kit can further include one or more lysis agents and/or permeabilization agents described herein.

In some embodiments, a kit further includes a device for applying pressure to a tissue for dispersion of the cells of the tissue into wells.

### EXAMPLES

### Example 1. Methods for Creating Spatial Capture Probes in Wells on a Substrate

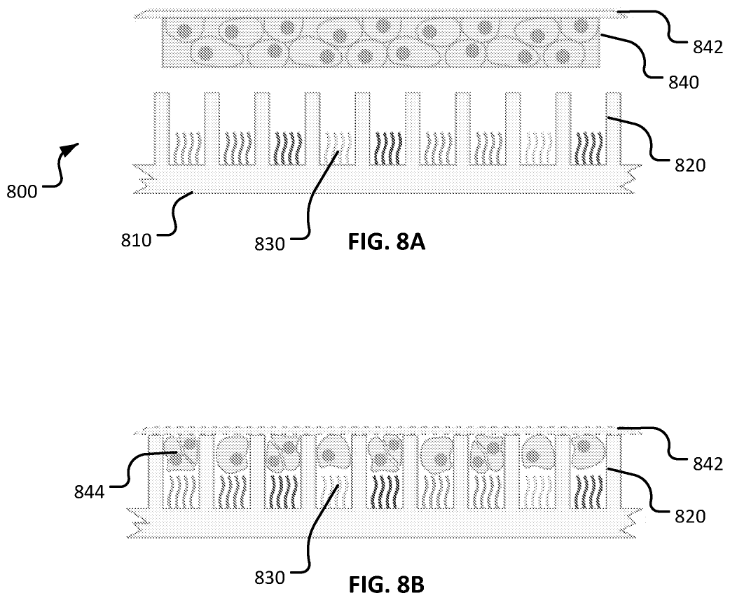

Exemplary substrates with wells in an array format are shown in FIGS. 7A and 7B. FIG. 7A shows a cutaway side view of a substrate 710 including wells (720) in an array format 700. Each well 720 is shown having a width 722 and depth 726, and each well 720 is separated from another well by a wall 724. Each well 720 of the array 700 is shown having the same width 722 and depth 726 and including a flat bottom portion. Additionally, each well 720 of the array 700 is separated by walls 724 of equal thickness. The wells 720 of the array 700 can be constructed into or onto a surface of the substrate 710 in any manner known in the art. For example, the wells 720 can be etched into the surface of the substrate using existing lithography techniques.

Each well 720 is shown to contain a plurality of spatially-barcoded capture probes 730. In general, the capture probes 730 comprise a cleavage domain, functional sequence, spatial barcode, and a capture domain. The functional sequence can be any optional sequence as described herein. The spatial barcode can be any barcode sequence as described herein. The capture domain can be an anchoring sequence designed to ensure that the capture domain hybridizes to the target analyte. The capture probes 730 of each individual well 720 are substantially the same, and the capture probes 730 between different wells 720 each have a unique spatial barcode to aid determination of the spatial location of the target analyte after capture.

The capture probes 730 of FIG. 7A are shown immobilized to the bottom surface of the well 720, though in some embodiments, the capture probes 730 can be immobilized to a side surface of the well 720 alone or in combination with capture probes on the bottom of the well. In general, the capture probes 730 can be immobilized on one or more surfaces of the well 720 (e.g., two or more, three or more).

FIG. 7B shows an oblique top-view of the exemplary well array 700 in or on the substrate 710. The array 700 is shown as a regular array, e.g., equal wall thickness 724 and center-to-center spacing 723, of eighteen wells 720 with hexagonal openings. In general, the well array 700 can extend to cover a portion of the substrate 710.

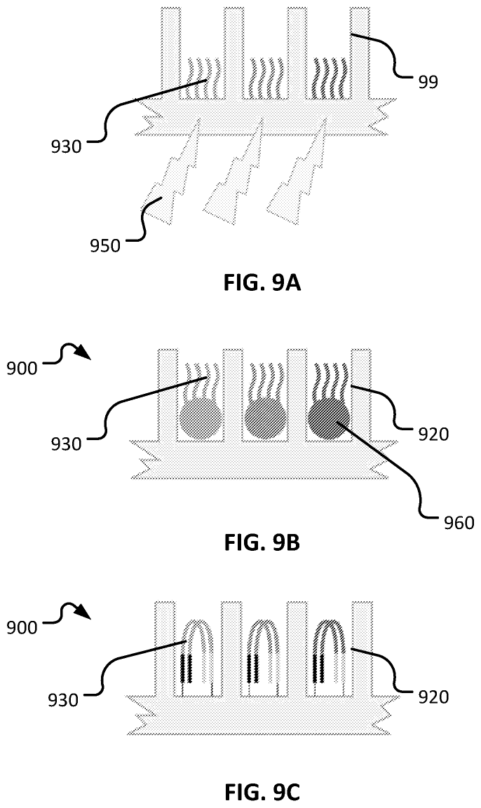

An exemplary workflow process for isolating a portion of a biological sample in the well array 700 is shown in FIGS. 8A-8B. As shown in FIG. 8A, a biological sample 840 is shown affixed (e.g., reversibly affixed) to a supportive backing 842. The biological sample can be any sample described herein. The supportive backing 842 can generally be a rigid or flexible backing capable of supporting the biological sample 840 during the procedure. In some embodiments, the backing 842 can be liquid-permeable. The biological sample 840 is positioned above the well array 800 with the backing 842 oriented away from the wells 820 and the biological sample 840 immediately adjacent to the top of the array.

The biological sample 840 is then pressed into the well array 800 as shown in FIG. 8B. The biological sample 840 is pressed into the array 800 through mechanical pressure guiding portions of the biological sample 840 into the individual wells 820. In some embodiments, the biological sample 840 can be pressed into the array 800 by the user with pressure applied by a hand or finger. In some embodiments, the biological sample 840 can be pressed into the array 800 with a user-operated device, e.g., a roller.

The pressure applied to the backing 842 can be sufficient to section the biological sample 840 into smaller samples using the openings of the array 800, thereby segmenting and dispersing the sample 840. In general, each well 820 can contain a single segment 844 of a biological sample and a plurality of wells can each contain a segment 844 of a biological sample. A segment 844 can be any portion of the biological sample 840 that is pressed into a well 820 and can constitute, for example, a cell of the biological sample 840, or more than one cell. FIG. 8B depicts each well 820 in the array 800 containing a segment 844 including between one and two exemplary cells of the biological sample 840. The segments 844 of the biological sample 840 and their contents are substantially contained within each well 820 and in proximity with the capture probes 830 upon lysis of the segments 844. In some embodiments, once the sample 840 has been segmented into the array 800, the backing 842 can optionally be removed. A buffer solution can be contacted to the backing 842. The buffer can permeate the liquid-permeable backing 842 and flow into each well 820, including wells 820 containing a segment of a biological sample 844. The buffer can include one or more lysis reagents capable of disrupting the cell wall or membrane of the segments 844 within each well 820. The lysis reagents can be any bioactive or chemical lysis reagent listed herein. The buffer containing lysis reagents can be allowed to contact the segments 844 for a duration of time, thereby lysing the cells of the segment 844, and releasing the analytes of interest from within the cells.

The analytes of interest interact with the capture domain of the capture probes 830 within each well 820 for an incubation period. The capture probes 830 are cleaved from the surface of the wells 820 through any cleavage means described herein. Library construction protocols are then performed to determine the spatial location of the bound analytes.

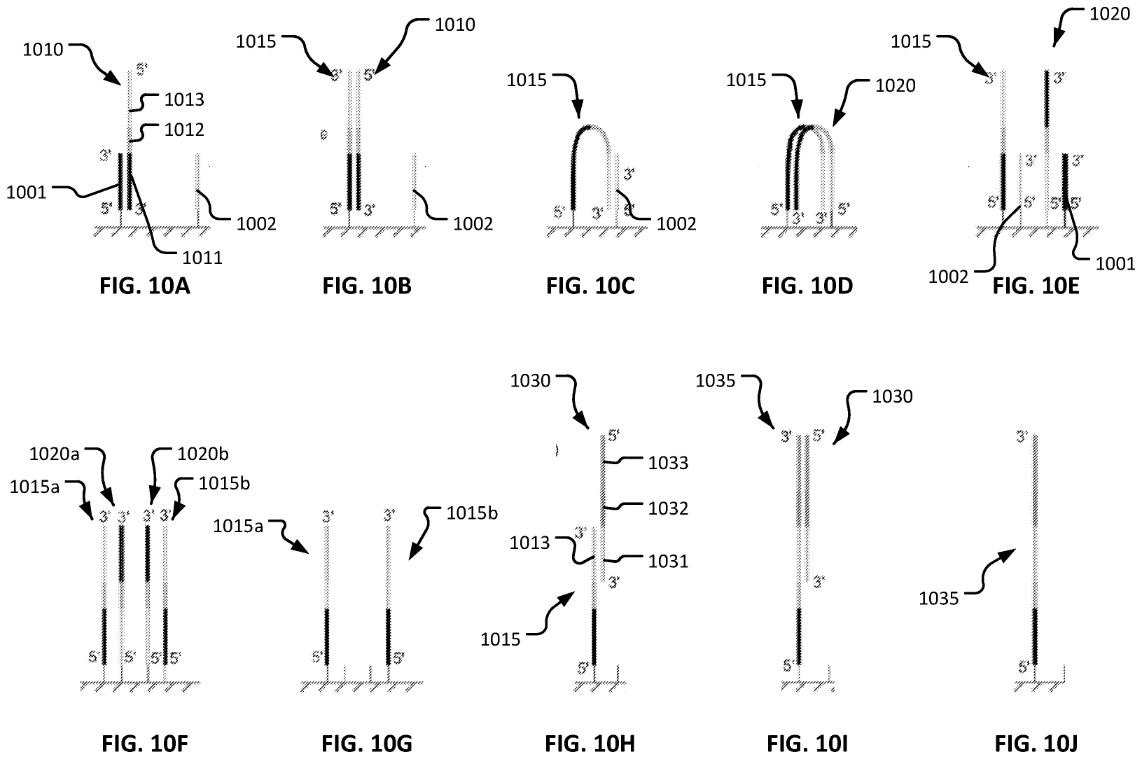

The capture probes of FIGS. 7 and 8 can be affixed to a surface of the wells using a number of methods. FIGS. 9A through 9C generally depict three exemplary methods of affixing the capture probes to a surface of the wells. FIG. 9A depicts the capture probes 930 constructed on the bottom surface of a well 920 using nucleotide photolithography. Briefly, nucleotide photolithography relies on building nucleic acid chains through single nucleotide additions using protective photolabile groups. Photolithographic chemical synthesis on a solid support can selectively synthesize probes directly on a surface of the array as known in the art.

Briefly, a covalent linker molecule on a one or more surfaces of a well 920 can include a photolabile protective group (PPG) on the exposed end. The PPG can be removed by light 950 of a wavelength specific to the PPG. For example, nitrobenzyl-based PPGs can be cleaved with light 950 of wavelength between 200 nm and 320 nm. The light 950 is directed through a photolithographic mask which selectively specifies which wells 920 are exposed. The light 950 removes the PPG from the linker molecule affixed to the surface of the well 920 thereby activating the linker molecules within the exposed wells 920 for combination with nucleotide groups in solution.

A solution containing nucleotides with a conjoined PPG group is added into the wells 920 and exposed linker molecules can bind with incoming protected nucleotides, adding a single PPG-protected nucleotide to the chain. Linkers that have not been exposed by light 950 exposure are not expected to bind with the incoming protected nucleotides. Unbound nucleotides are removed through buffer exchange and the process repeated with new rounds of light 950 exposure and protected nucleotide addition, thereby constructing nucleic acid chains on the linker molecules of a known and specific sequence.

Alternatively, FIG. 9B depicts a process wherein capture probes 930 can be disposed within wells 920 while affixed to beads 960. In general, capture probes 930 can be affixed to the surface of beads 960 via covalent linkage with the surface of the bead 960 as described herein. For example, the capture probes 930 can be affixed to the surface of the bead 960 through the use of an avidin or streptavidin linker. The beads 960 can be composed of any material disclosed herein, but in general, dissolvable hydrogel materials can allow for the placement of affixed capture probes 930 within specific wells to be bound to the surfaces of the well 920.

In general, a buffer containing beads 960 with affixed capture probes 930 is flowed onto the array 900. The diameter of the beads 960 can be about 50% to 80% of the depth of the well 920 (e.g., 55% to 80%, 60% to 80%, 65% to 80%, 70% to 80%, 75% to 80%, 50% to 75%, 50% to 70%, 50% to 65%, 50% to 60%, or 50% to 55%). In this manner, a single bead 960 can be deposited into a well 920. The capture probes 930 affixed to the surface of a bead 960 include a common spatial barcode such that each bead 960 spatially identifies a single well. The beads 960 can be dissolved and the capture probes 930 released and affixed to a surface of the well 920.

As a further alternative, capture probes 930 can be constructed within the wells 920 of the array 900 using bridge amplification, as shown in FIG. 9C. Generally, nucleic acid primers are first attached to one or more surfaces of the wells. In some embodiments, two primers can be used such as a forward primer and a reverse primer, wherein the first primer sequence can specifically bind to a first adapter sequence, and the second primer sequence can specifically bind to a second adapter sequence. Further details of bridge amplification can be found in FIGS. 10A-10J.

FIG. 10A depicts the first primer sequence 1001 bound to a first adapter sequence 1010 and the second primer sequence 1002 being unbound. In general, the adapter sequence 1010 can include additional sequences for nucleic acid amplification. For example, the adapter sequence 1010 of FIG. 10A includes a binding sequence 1011 complimentary to the first primer sequence 1001, a spatial barcode sequence 1012, and a linker sequence 1013 complementary to the second primer sequence 1002. Shown in FIG. 10B, the first primer sequence 1001 is then extended using reverse transcription into a first complementary DNA (cDNA) 1015 strand, complementary to the adapter sequence 1010. The adapter sequence 1010 is then denatured from the extended cDNA 1015. Buffer is flowed across the surface of the well. The linker sequence 1013 of the extended cDNA 1015 contacts the second primer sequence 1002 and is specifically bound, thereby creating a “bridge” structure as in FIG. 10C.

Further shown in FIG. 10D, the bridged cDNA 1015 is used as a polymerization template in extending the second primer sequence 1002 into a second cDNA 1020 with the complimentary sequence of the first cDNA 1015. Following polymerization, the first cDNA 1015 and second cDNA 1020 are affixed to a surface of the well via respective 5′ ends and bound through complementary base pairing. The first cDNA 1015 and second cDNA 1020 are denatured through any means described herein.

Referring now to FIG. 10E, following denaturation of the first cDNA 1015 and the second cDNA 1020, the second cDNA 1020 shares the same sequence as the adapter sequence 1010 in reverse order, e.g., having the linker sequence 1013 proximal to the surface of the well. The first cDNA 1015 and the second cDNA 1020 can provide new extended substrates for repeated rounds of bridge amplification. In the same manner as above, the first cDNA 1015 can be bridged to a second primer sequence 1002 and the second cDNA 1020 bridged to a first primer sequence 1001. The first cDNA 1015 and the second cDNA 1020 are replicated via polymerization to produce additional first cDNA and second cDNA strands. FIG. 10F depicts replicated first cDNA strands 1015a and 1015b and second cDNA strands 1020a and 1020b.

Following several rounds of bridge amplification to achieve the necessary density of first cDNA 1015 strands, the second cDNA 1020 sequences can be removed, e.g., cleaved, from the surface of the well. FIG. 10G depicts remaining first cDNA strands 1015a and 1015b.

As shown in FIG. 10H, a second adapter sequence 1030 can be added to the solution. The second adapter sequence 1030 includes a second linker sequence 1031 complementary to the first linker sequence 1013, a unique molecular identifier (UMI) 1032, and an analyte capture sequence or domain 1033. The second adapter sequence 1030 binds to the first linker sequence 1013 of one of the plurality of first strand cDNAs 1015.

Referring to FIG. 10I, the second adapter sequence 1030 is used as a reverse transcription template to extend the plurality of first strand cDNAs 1015 to form extended cDNAs 1035 that include the additional sequences moieties, e.g., UMI, analyte capture sequence, that were included in the second adapter sequence 1030.

Depicted in FIG. 10J, the second adapter sequences 1030 are denatured from the plurality of extended first cDNAs 1035 and removed from the solution through dilution or buffer exchange. The plurality of extended first cDNA 1035 strands are capture probes capable of binding to any target analyte containing sequences complementary to the analyte capture sequence 1033 following sample lysis.

### Example 2. Electrophoretic Methods for Well Based Spatial Arrays

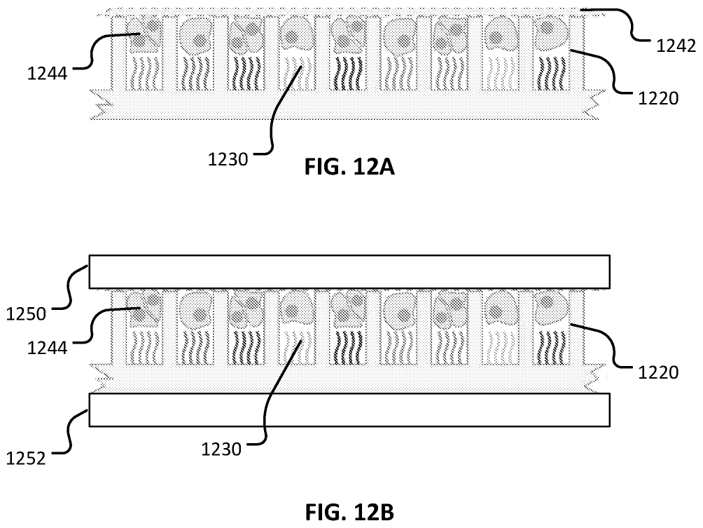

In some embodiments, an electric field can be applied to the well array to motivate target analytes toward the capture probes in the micro wells. As shown in FIG. 11A, a biological sample 1140 is shown affixed (e.g., reversibly affixed) to a supportive backing 1142. The biological sample can be any sample described herein. The supportive backing 1142 can generally be a rigid or flexible backing capable of supporting the biological sample 1140 during the procedure. In some embodiments, the backing 1142 can be liquid-permeable. The biological sample 1140 is positioned above the well array 1100 with the backing 1142 oriented away from the wells 1120 and the biological sample 1140 immediately adjacent to the top of the array.

The biological sample 1140 is contacted to the upper surface of the well array 1100 as shown in FIG. 11B. A positive electrode 1150 is contacted to a side of the biological sample 1140 and a negative electrode 1152 is contacted to a side of the well array 1100. In some embodiments, the well array 1100 includes a conductive surface opposing the wells 1120 to facilitate electric conduction. For example, the well array 1100 surface opposing the wells 1120 can be coated in a conductive substance, e.g., a metal, or a semi-conductor.

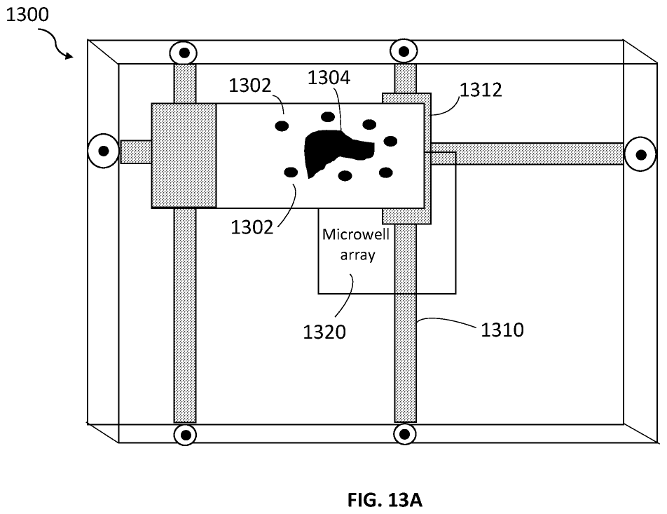

In some embodiments, the biological sample 1140 can be sectioned and an electric field applied. FIG. 12A shows a biological sample 1240 segmented into a plurality of smaller samples and dispersed into the wells 1220. As previously described with reference to FIG. 8B, each well 1220 in the array 1200 contains a segment 1244 including between one and two exemplary cells of the biological sample 1240. The segments 1244 of the biological sample 1240 and their contents are substantially contained within each well 1220 and in proximity with the capture probes 1230 upon lysis of the segments 1244.

A positive electrode 1250 is contacted to a side of the biological sample 1240 and a negative electrode 1252 is contacted to a side of the well array 1200. An electric field is created between the positive electrode 1250 and the negative electrode 1252. Analytes from the biological sample 1240 migrate to the capture probes 1230.

In some embodiments, the positive electrode 1250 is contacted to a side of the well array 1200 and the negative electrode 1252 is contacted to a side of the biological sample 1240 depending on the charge of the target analyte.

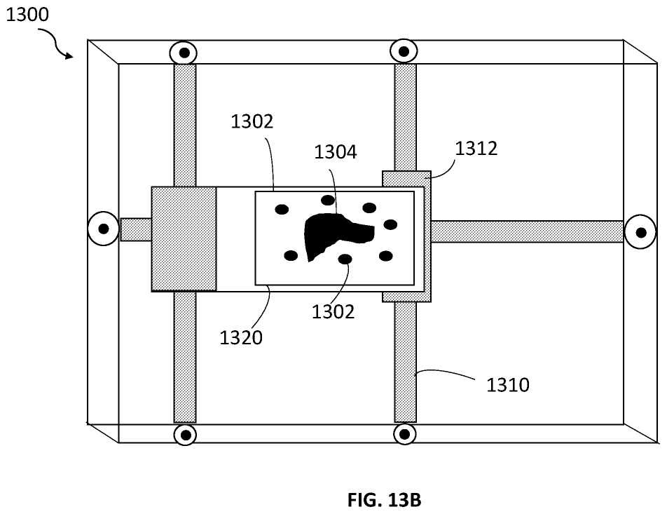

In some embodiments, the biological sample 1240 is positioned adjacent the array 1200 by an actuator system. The actuator system can facilitate the alignment of the biological sample 1240 adjacent the wells 1220 of an array 1200. Referring now to FIGS. 13A and 13B, an exemplary actuator system 1300 having actuators 1310 and a slide tray 1312 is shown. A substrate 1302 having a biological sample 1304 is placed into the slide tray 1312. The actuator system 1300 operates the actuators 1310 to arrange the biological sample 1304 adjacent the wells of the microwell array 1320. The actuators 1310 operate to position the biological sample 1304 in the vertical, or horizontal, direction such that the biological sample 1304 is brought into contact with the wells of the microwell array 1320.

Any method of migrating the target analytes to the capture probes is then employed to facilitate library preparation.

1.-40. (canceled)

41. A method of determining a location of an analyte in a biological sample, the method comprising:
(a) providing a substrate comprising a plurality of wells, wherein a well of the plurality of wells comprises:
(i) one or more lysis agent(s);
(ii) a surface comprising a plurality of capture probes, wherein a capture probe of the plurality of capture probes comprises a spatial barcode and a capture domain, wherein the plurality of capture probes are attached to a surface of the wells using bridge amplification prior to step (a); and
(iii) a plurality of analyte capture agents, wherein an analyte of the plurality of analyte capture agents comprises an analyte binding moiety, an analyte binding moiety barcode, and an analyte capture sequence;
(b) disposing the biological sample into the plurality of wells;
(c) releasing the analyte from the biological sample, wherein the analyte is specifically bound by the analyte binding moiety of the analyte capture agent, and the analyte capture sequence of the analyte capture agent is bound by the capture domain of the capture probe; and
(d) determining (i) a sequence corresponding to the analyte binding moiety barcode or a complement thereof, and (ii) a sequence corresponding to the spatial barcode or a complement thereof, and using the sequences of (i) and (ii) to determine the location of the analyte in the biological sample.

42. The method of claim 41, wherein the substrate comprises about 1 million wells to about 5 million wells.

43. The method of claim 41, wherein each well of the plurality of wells is a flat-bottomed well or a round-bottomed well.

44. The method of claim 41, wherein each well of the plurality of wells comprises a perimeter that is hexagonally-shaped, heptagonally-shaped, octagonally-shaped, pentagonally-shaped, square-shaped, or circularly-shaped.

45. The method of claim 41, wherein each well of the plurality of wells comprises substantially the same perimeter length.

46. The method of claim 41, wherein each well of the plurality of wells do not comprise substantially the same perimeter length.

47. The method of claim 41, wherein each well of the plurality of wells comprises one or more of:
an opening and/or a bottom surface having an average diameter of about 3 μm to about 7 μm;
an opening and/or a bottom surface having an area of about 12 μm² to about 30 μm², and
a depth of about 10 μm to about 35 μm.

48.-54. (canceled)

55. The method of claim 41, wherein the plurality of wells comprises a geometric center-to-center spacing between adjacent wells of about 7.0 μm to about 20 μm.

56.-57. (canceled)

58. The method of claim 41, wherein the plurality of capture probes is disposed on:
a bottom surface of the well;
one or more side surface(s) of the well; or
a bottom surface of the well and on one or more side surface(s) of the well.

59.-60. (canceled)

61. The method of claim 41, wherein the disposing in step (b) is performed using pressure applied using a roller or a stamping device.

62.-67. (canceled)

68. The method of claim 41, wherein the method further comprises, prior to step (b), one or more of fixing, staining, and/or imaging the biological sample.

69. (canceled)

70. The method of claim 41, wherein the biological sample is a tissue sample.

71. The method of claim 70, wherein the tissue sample is a fresh, frozen tissue sample or a fixed tissue sample.

72. (canceled)

73. The method of claim 70, wherein the tissue sample is a formalin-fixed paraffin-embedded (FFPE) tissue sample.

74. The method of claim 41, wherein step (d) comprises extending an end of the capture probe using the analyte binding moiety barcode as a template.

75. The method of claim 41, wherein step (d) comprises sequencing (i) the sequence corresponding to the analyte binding moiety barcode or the complement thereof, and (ii) the sequence corresponding to the spatial barcode or the complement thereof.

76. The method of claim 75, wherein the sequencing is high throughput sequencing.

77. The method of claim 41, wherein the analyte binding moiety is an antibody or an antigen-binding fragment thereof.

78. The method of claim 41, wherein the analyte is a protein

79. The method of claim 78, wherein the protein is an intracellular protein.

80.-124. (canceled)
